(19) United States
(12) Patent Application Publication
(10) Pub. No.: US 2025/0284747 A1
(43) Pub. Date: Sep. 11, 2025

(54) CONTINUITY AND DISASTER RECOVERY SOLUTION FOR PATIENTS UNDER TREATMENT IN RADIATION ONCOLOGY

(71) Applicant: Ohio State Innovation Foundation, Columbus, OH (US)

(72) Inventors: Dominic DICOSTANZO, Columbus, OH (US); Nilendu GUPTA, Columbus, OH (US); Charles COUGHLIN, Galena, OH (US); Matthew TENNEY, Columbus, OH (US)

(21) Appl. No.: 18/858,883

(22) PCT Filed: Apr. 21, 2023
(86) PCT No.: PCT/US2023/019333

§ 371 Date: Oct. 22, 2024

Related U.S. Application Data
(60) Provisional application No. 63/334,148, filed on Apr. 24, 2022.

(51) Int. Cl. G06F 16/93; G16H 10/60; G16H 15/00

(52) U.S. Cl. CPC G06F 16/93; G16H 10/60; G16H 15/00

(57) ABSTRACT

Systems and methods that automate the backup of specific patient related information to network attached, but segregated device. Each patient's information that is required for treatment (DICOM files) are automatically queried from the EHR databased and transferred using DICOM transactions to the workstation through specified TCP ports. The software automatically organizes and stores the files on a secure/encrypted USB drive. The software then leverages web-based application programming interfaces (APIs) to pull patient documents that are relevant for treatment. Finally, a treatment summary report is generated and stored along-side the EHR documents and DICOM files. In the event of a failure, the USB device can be removed and plugged into the treatment delivery devices to allow for treatment of patients under care.

### CROSS-REFERENCE TO RELATED APPLICATION

This application claims priority to U.S. Provisional Patent Application No. 63/334,148, filed Apr. 24, 2022, entitled “CONTINUITY AND DISASTER RECOVERY SOLUTION FOR PATIENTS UNDER TREATMENT IN RADIATION ONCOLOGY,” which is incorporated herein by reference in its entirety.

### FIELD OF THE INVENTION

The present disclosure relates to disaster recovery and more specifically to a system for saving patent data and treatment plans in a record and verify system to a secured file storage that may be used by a radiotherapy treatment delivery device in the event of an outage of the record and verify system or outages to the network connection between the radiotherapy treatment delivery device and the record and verify system.

### BACKGROUND

Radiotherapy delivery systems, such as TRUEBEAM (available from Varian Medical Systems, Inc.), use patient records and treatment plans to provide cancer treatment to patients. Typically, patient records and treatment plans are maintained in a record and verify system. The records and treatment plans are communicated from record and verify system to the radiotherapy delivery system over a local area network in advance of a patient's treatment. Once treatment is completed by the radiotherapy delivery system, the patient records and treatment plans are transmitted back to the record and verify system.

Thus, because the radiotherapy delivery system depends on the record and verify system being available, if there is an outage of the record and verify system or the local area network that connects to the record and verify system, the radiotherapy delivery system is unable to provide treatment to patients. For those patients needing critical care, this could have catastrophic consequences for those patients. Thus, there is a need to ensure that patient data and treatment plans are available to the radiotherapy delivery system when the record and verify system is unavailable.

### SUMMARY

Accordingly, the present disclosure directed to a method for continuity and disaster recovery in an oncology treatment system. The method includes querying a treatment planning system for a list of medical record numbers (MRN); for each retrieved MRN, retrieving a plan unique ID (UID); using the MRN and UID to query the treatment planning system initiating DICOM transactions; exporting patient and associate treatment data using the DICOM transactions; and storing the exported patient and associated treatment data on a secured file storage.

The foregoing illustrative summary, as well as other exemplary objectives and/or advantages, and the manner in which the same are accomplished, are further explained within the following detailed description and its accompanying drawings.

### BRIEF DESCRIPTION OF THE DRAWINGS

A detailed description of certain aspects of the present disclosure in accordance with various example implementations will now be provided with reference to the accompanying drawings. The drawings form a part hereof and show, by way of illustration, specific implementations and examples. In referring to the drawings, like numerals represent like elements throughout the several figures.

FIG. 1 illustrates an example environment in which the present disclosure may implemented;

FIG. 2A illustrates an example process that may be implemented in the environment of FIG. 1;

FIG. 2B illustrates another example process that may be implemented in the environment of FIG. 1; and

FIG. 3 is an example computing device that may execute instructions to perform the process of FIG. 2.

### DETAILED DESCRIPTION

The present disclosure is directed to a system that automates the backup of specific patient related information to network attached, but segregated device. Each patient's information that is required for treatment (DICOM files) are automatically queried from the EHR databased and pulled using DICOM transactions to the workstation through specified TCP ports. The software automatically organizes and stores the files on a secure/encrypted USB drive. The software then leverages web-based application programming interfaces (APIs) to pull patient documents that are relevant for treatment. Finally, a treatment summary report is generated and stored along-side the EHR documents and DICOM files. In the event of a failure, the USB device can be removed and plugged into the treatment delivery devices to allow for treatment of patients under care.

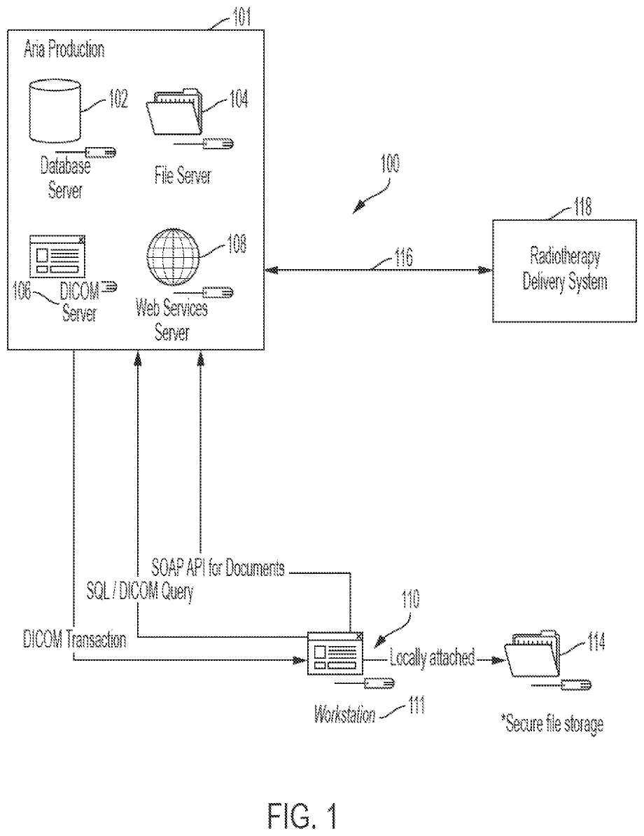

FIG. 1 illustrates an example environment 100 in which the present disclosure may be implemented. The environment 100 includes a record and verify system (e.g., ARIA Oncology Information System, available from Varian Medical Systems, Inc.) 101 having a database 102 that store patient data and treatment schedules, a file system 104 (e.g., a Common Internet File System (CIFS) to provide shared access to files), a DICOM interface 106 that supports the transfer of images and plan data using the DICOM protocol, and a web server 108 that supports Simple Object Access Protocol (SOAP) API request and responses.

A workstation 110 may be a hardened computing device that meets or exceeds the Center for Internet Security (CIS) Level 2 environmental benchmark. For example, the workstation 110 may be a Dell Precision 5820 Tower running Windows 10 Pro for Workstations (or higher). The workstation 110 executes a scheduled task a customizable time point to implement the processes described herein. A secured file storage 114 may be encrypted USB drive (or other removable media) having hardware encryption. A radiotherapy delivery system 118 (e.g., treatment delivery device), such as TRUEBEAM, provides cancer treatment to patients, and is adapted to receive patient data and treatment plans from the record and verify system 101.

In the environment 100, the radiotherapy delivery system 118 would normally retrieve patient data and treatment plans from the record and verify system 101 over a network communication link 116, utilize that patient data and treatment plans to treat patients under care, and return updated and verified patient data and treatment plans to the record and verify system 101 over the network communication link 116. However, a failure of the network communication link 116 and/or the record and verify system 101 would result in the radiotherapy delivery system not having critical patient information and treatment plans needed to treat cancer patients under care.

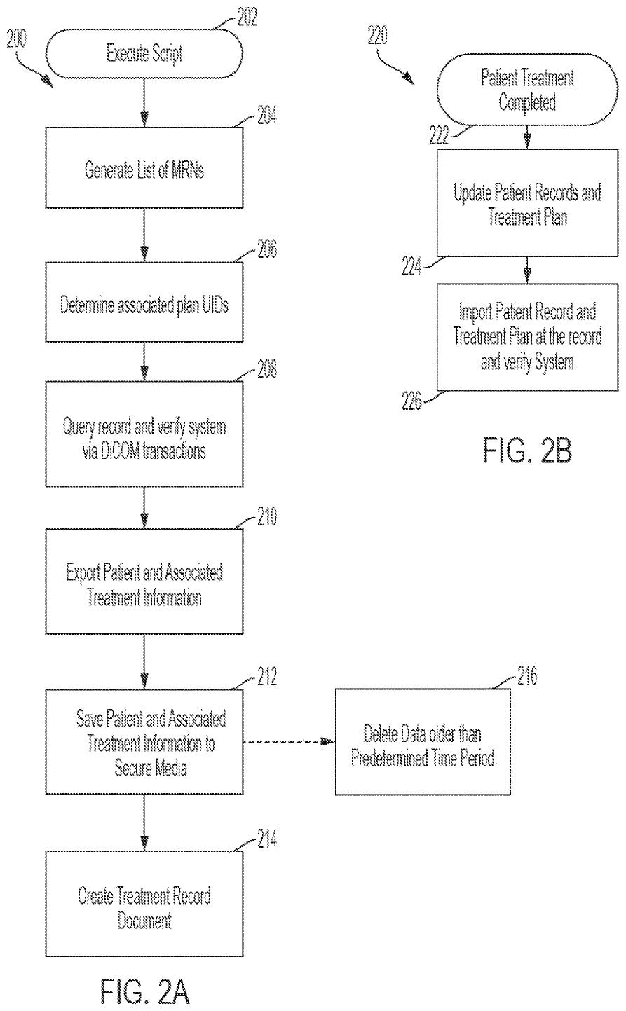

Thus, to prevent the scenario above, FIG. 2A, illustrates an example process 200 in accordance with the present disclosure. At 202, execution of a script to retrieve patient data begins. For example, each night at 2:00 AM, the script is run to search a treatment schedule for the current calendar day for all patients with a scheduled treatment activity. A schedule of the patients may also be created and stored within the folder for each treatment device (e.g., TRUEBEAM). The report may contain the patient MRN, name, appointment start date and time, appointment end date and time, any notes for the activity, and the appointment status. At 204, a list of Medical Record Numbers (MRN) is generated. At 206, for each MRN, a plan unique ID (UID) for each patient's treatment is determined. These may include approved plans with creation date within the previous 90 days in an Active Course. Using the UID for the patient's Plan, each CT, Structure Set, and DRR UID is captured. At 208, the list of MRN and associated UIDs required for treatment is provided to a software solution that queries the Aria environment via DICOM transactions. To secure the query process, the workstation 110 is configured to accept only DICOM File transactions from the DICOM daemon in the production environment 106. At 210, each patient and the associated treatment information is transferred using DICOM network transactions using customized software and the resulting files are stored on the network segregated workstation 111. The DICOM endpoint 112 located on workstation 111 will point to the secured file storage 114 which will be locally attached using, e.g., a USB port of the workstation 110. The data may be stored in a separate directory for each day in the following folder structure: YYYYMMDD\MRN\.

At 212, patent data and treatment information are saved to the secured file storage. For example, script may be run on the workstation 111 that will capture all documents for the patients whose DICOM data was transferred. Using the list of MRNs, the script will query a Documents API exposed by the web server 108 and transfer each document (e.g., those less than 365 days old) for each patient. The documents may be stored in the following folder structure: YYYYMMDD\MRN\.

At 214, a treatment record document is created. This document may be a Microsoft Word document that presents a paper chart that can be used for treatment and reconciliation of data that is imported to the record and verify system 101 in accordance with the process 220 described below with reference to FIG. 2B. In addition, the treatment record document is unique snapshot of the patient that otherwise does not exist as an electronic health record (EHR).

Optionally, at 216, data older than a predetermined period of time may be deleted. For example, a cleanup script may be run on the workstation 111 each night that deletes all data (DICOM and Documents) greater than a predetermined period of time (e.g., 7 days) to free storage.

The data stored on the secured file storage 114 may be provided to the radiotherapy delivery system 118 such that patients under care may receive treatment when the record and verify system 101 and or communication network link 116 are not available. In particular, some radiotherapy delivery systems 118 allow for patient treatment using a flat files rather than DICOM network transactions. In this mode, patient DICOM information is stored on treatment delivery system 118 local storage or accessible network share. In accordance with the present disclosure, this feature may be utilized by inserting the secured file storage 114 into an appropriate port on the radiotherapy delivery device 118 to transfer the relevant treatment information the treatment delivery device 118 local storage, transfer the data over a network connection between the workstation 110 and the treatment delivery device, or a combination of both.

With reference to FIG. 2B, there is illustrated another example process 220 in accordance with the present disclosure. At 222, the patient treatment is completed at the radiotherapy delivery device. At 224, the radiotherapy device updates the patient records and treatment plan to reflect the completed treatment. In particular, the patient data and treatment plan are recorded to the secured file storage 114 and verified. At 226, the recorded in verified patient data and treatment plan is imported into the record and verify system 101.

During a downtime scenario when the record and verify system is unavailable, a paper record will be used to record patient treatment (e.g., using the treatment record document). Once the record and verify system becomes available and is electronically updated to include the patent treatment, the paper record will be used to reconcile the electronic data.

With reference to the processes 202 and 220, several cybersecurity concerns may be addressed by using the following:
Perform processes using a service account with extremely limited (singular right to write to singular directory);
Prevent other users from writing to that specific directory
Limit rights to only allow on .dcm, .docx, .xlsx, and .pdf files

### Example Environment

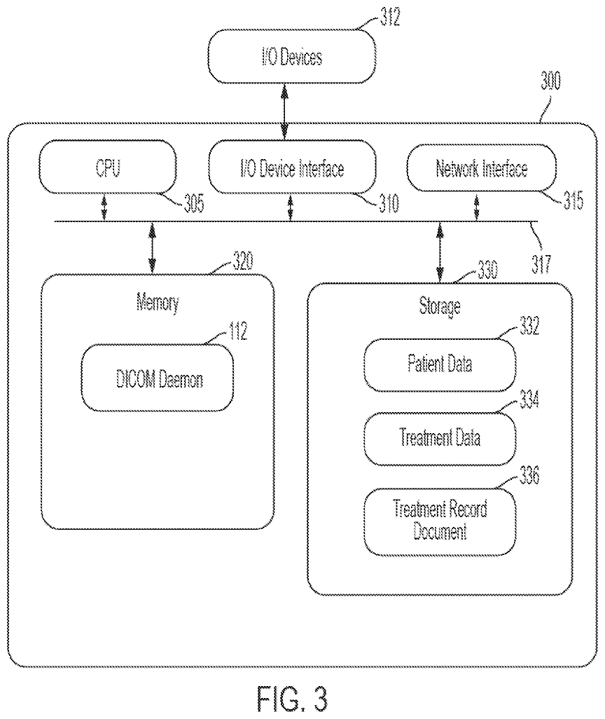

FIG. 3 illustrates examples of computers 300 that may include the kinds of software programs, data stores, and hardware that can implement event message processing, context determination, notification generation, and content delivery, as described above according to certain embodiments. As shown, the computing system 300 includes, without limitation, a central processing unit (CPU) 305, a network interface 315, a memory 320, and storage 330, each connected to a bus 317. The computing system 300 may also include an I/O device interface 310 connecting I/O devices 312 (e.g., keyboard, display and mouse devices) to the computing system 300. Further, the computing elements shown in computing system 300 may correspond to a physical computing system (e.g., a system in a data center) or may be a virtual computing instance executing within a computing cloud.

The CPU 305 retrieves and executes programming instructions stored in the memory 320 as well as stored in the storage 330. The bus 317 is used to transmit programming instructions and application data between the CPU 305, I/O device interface 310, storage 330, network interface 315, and memory 320. Note, CPU 305 is included to be representative of a single CPU, multiple CPUs, a single CPU having multiple processing cores, and the like, and the memory 320 is generally included to be representative of a random-access memory. The storage 330 may be a disk drive or flash storage device. Although shown as a single unit, the storage 330 may be a combination of fixed and/or removable storage devices, such as fixed disc drives, removable memory cards, optical storage, network attached storage (NAS), or a storage area-network (SAN).

Illustratively, the memory 320 includes the workstation 111, which is discussed in greater detail above. Further, storage 330 includes, patient data 332, treatment data 334 and treatment record document 336, all of which are also discussed in greater detail above.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although certain implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be affected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims. The present disclosure is capable of other implementations and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary implementations include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example implementations, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

We claim:

1. A method for continuity and disaster recovery in an oncology treatment system, comprising:
querying a record and verify system for a list of medical record numbers (MRN);
for each retrieved MRN, retrieving a plan unique ID (UID);
using the MRN and UID to query the record and verify system using DICOM transactions;
exporting patient and associate treatment data using the DICOM transactions; and
storing the exported patient and associated treatment data on a secured file storage.

2. The method of claim 1, further comprising retrieving UIDs created within a predetermined period of time.

3. The method of claim 1, further comprising accepting only DICOM transactions from a DICOM daemon in a predetermined environment.

4. The method of claim 1, wherein the secured file storage is locally attached to a workstation.

5. The method of claim 4, further comprising periodically executing a script to delete data older than a predetermined period of time.

6. The method of claim 1, further comprising executing a script to capture documents to the secured file storage by using a list of MRNs to query a Documents API exposed by a web server and transfer each document for each patient.

7. The method of claim 6, further comprising capturing associated treatment data that is newer than a predetermined date.

8. The method of claim 1, further comprising creating a treatment record document.

9. The method of claim 8, further comprising generating a paper treatment record document that is used to reconcile data electronic data stored in the secured file storage.

10. The method of claim 1, further comprising providing the secured file storage to a radiotherapy delivery system.

11. A disaster recovery in an oncology treatment system, comprising:
a memory comprising computer-executable instructions; and
a processor configured to execute the computer-executable instructions and cause the computer system to perform a method of providing realtime contextual event notifications, the computer system executing instructions to:
query a record and verify system for a list of medical record numbers (MRN);
for each retrieved MRN, retrieve a plan unique ID (UID);
use the MRN and UID to query the record and verify system using DICOM transactions;
export patient and associate treatment data using the DICOM transactions; and
store the exported patient and associated treatment data on a secured file storage.

12. The system of claim 11, the processor further comprising instructions to retrieve UIDs created within a predetermined period of time.

13. The system of claim 11, the processor further comprising instructions to accept only DICOM transactions from a DICOM daemon in a predetermined environment.

14. The system of claim 11, wherein the secured file storage is locally attached to a workstation.

15. The system of claim 14, the processor further comprising instructions to execute a script to delete data older than a predetermined period of time.

16. The system of claim 11, the processor further comprising instructions to execute a script to capture documents to the secured file storage by using a list of MRNs to query a Documents API exposed by a web server and transfer each document for each patient.

17. The system of claim 16, the processor further comprising instructions to capture associated treatment data that is newer than a predetermined date.

18. The system of claim 11, the processor further comprising instructions to create a treatment record document.

19. The system of claim 18, the processor further comprising instructions to generate a paper treatment record document that is used to reconcile data electronic data stored in the secured file storage.

20. The system of claim 11, the processor further comprising instructions to provide the secured file storage to a radiotherapy delivery system.
